(19) United States
(12) Patent Application Publication
(10) Pub. No.: US 2013/0035338 A1
(43) Pub. Date: Feb. 7, 2013

(54) EYELID TREATMENT

(71) Applicant: Gordon Tang, Bellevue, WA (US)

(72) Inventor: Gordon Tang, Bellevue, WA (US)

(21) Appl. No.: 13/567,931

(22) Filed: Aug. 6, 2012

Related U.S. Application Data
(60) Provisional application No. 61/515,815, filed on Aug. 5, 2011.

(51) Int. Cl. A61K 31/5575; A61K 31/382; A61K 31/5377; A61P 21/00

(52) U.S. Cl. 514/236.2; 514/530; 514/622; 514/432

(57) ABSTRACT

The current application is directed topical treatments that improve facial appearance by causing eyelids to naturally open more widely, or be more fully raised, as well as to treatment of ptosis and other eyelid-related conditions. These topical treatments include one or more of travoprost, dorzolamide hydrochloride, timolol maleate, unoprostone, latanoprost, bimatoprost, and related compounds currently used for treating glaucoma and ocular hypertension.

### CROSS-REFERENCE TO RELATED APPLICATION

This application claims the benefit of Provisional Application No. 61/515,815, filed Aug. 5, 2011.

### TECHNICAL FIELD

The present invention is related to eyelid-related ailments and conditions and, in particular, to treatment of ptosis and other eyelid-related conditions as well as to cosmetic applications to enhance eyelid appearance.

### BACKGROUND

Drooping eyelids, a condition referred to as “ptosis,” is an often age-related condition that can also be caused by diseases and injuries or by congenital defects, can lead to an undesirable appearance as well as restricted vision. Often, an individual may not have ptosis, but may nonetheless desire, for appearance and cosmetic reasons, for his or her eyelids to appear to open more fully to enhance facial appearance. Currently, surgery is most commonly employed to treat ptosis and to cosmetically enhance eyelid appearance.

### SUMMARY

Various embodiments of the present invention are directed topical treatments that improve facial appearance by causing eyelids to naturally open more widely, or be more fully raised, as well as to treatment of ptosis and other eyelid-related conditions.

### BRIEF DESCRIPTION OF THE DRAWINGS

FIG. 1 shows the structural formula for travoprost.

FIG. 2 shows the structural formula for dorzolamide hydrochloride.

FIG. 3 shows the structural formula for timolol maleate.

FIG. 4 shows the structural formula for unoprostone.

FIG. 5 shows the structural formula for latanoprost.

FIG. 6 shows the structural formula for bimatoprost.

### DETAILED DESCRIPTION

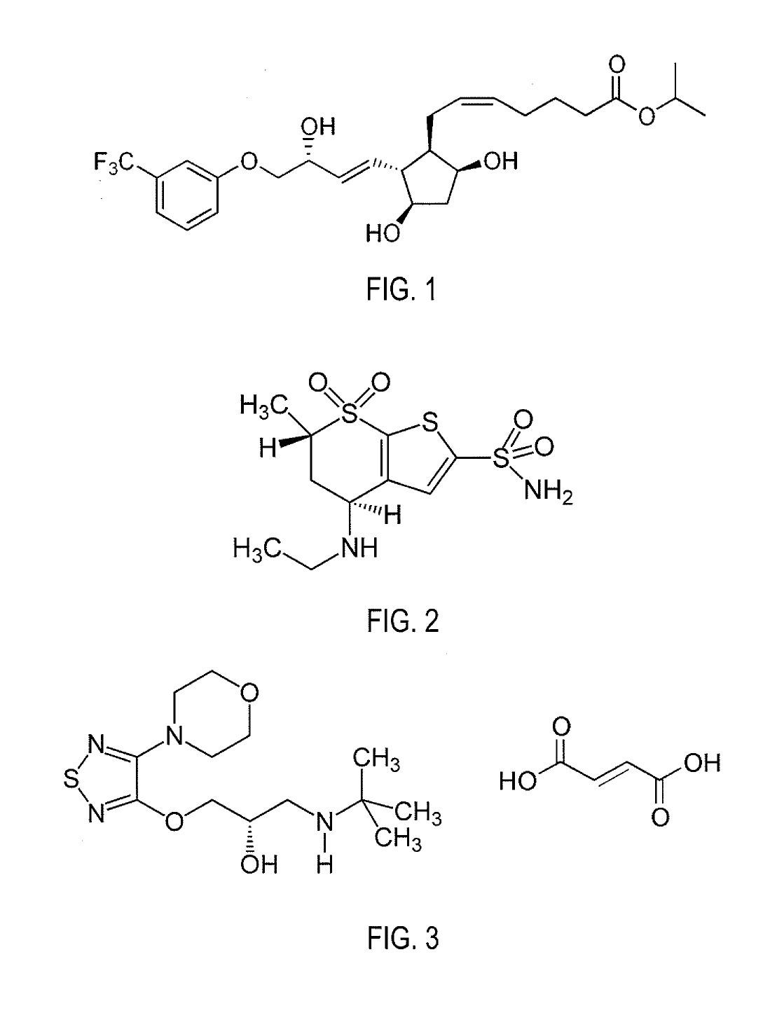

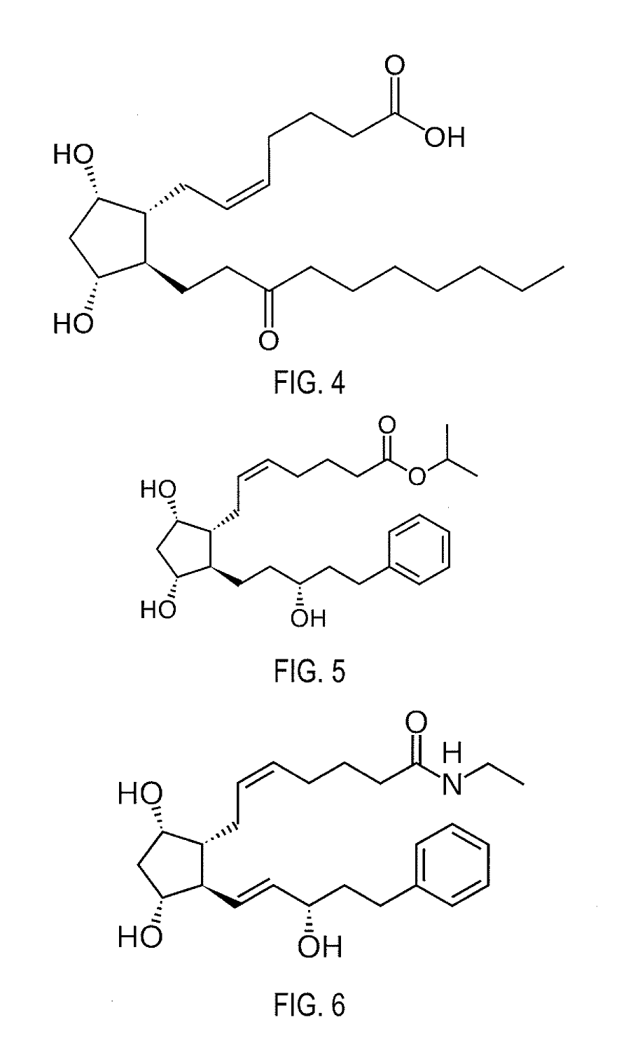

Many medications are currently used to treat glaucoma and ocular hypertension. These include: (1) Travatn®, which contains travoprost, propan-2-yl 7-[3,5-dihydroxy-2-[3-hydroxy-4-[3-(trifluoromethyl)phenoxy]-but-1-enyl]-cyclopentyl]hept-5-enoate, a synthetic prostaglandin F analogue, the structural formula for which is shown in FIG. 1; (2) Cosopt®, which includes dorzolamide hydrochloride,(4S,6S)-2-ethylamino-4-methyl-5,5-dioxo-5λ⁶, 7dithiabicyclo [4.3.0] nona-8,10-diene-8-sulfonamide, the structural formula for which is shown in FIG. 2; (3) timolol maleate, (S)-1-(tert-butylamino)-3-[(4-morpholin-4-yl-1,2,5-thiadiazol-3-yl)oxy]propan-2-ol, the structural formula for which is shown in FIG. 3; (4) Rescula®, which includes unoprostone isopropyl, isopropyl (Z)-7-[(1R,2R,3R,5S)-3,5-dihydroxy-2-(3-oxodecyl)cyclopentyl]hept-5-enoic acid, the structural formula for which is shown in FIG. 4; (5) Xalatan®, which contains latanoprost, isopropyl (Z)-7-[(1R,2R,3R,5S)-3,5-dihydroxy-2-[(3R)3-hydroxy-5-phenylpentyl]-cyclopentyl]hept-5-enoate. the structural formula for which is shown in FIGS. 5; and (6) Lumigan®, which contains bimatoprost, 7-[3,5-dihydroxy-2-(3-hydroxy-5-phenyl-pent-1-enyl)-cyclopentyl]-N-ethyl-hept-5-enamide, the structural formula for which is shown in FIG. 6. These compounds are normally suspended or dissolved in sterile, isotonic, buffered aqueous solutions for use as eye drops, and effect lowering of the internal pressure within the eye, or intraocular pressure.

Recently, the current inventor has discovered that travoprost, dorzolamide hydrochloride, timolol maleate, unoprostone, latanoprost, and bimatoprost can be topically applied, alone or in combination, to the eyelids in order to cause the eyelids to retract more completely, thus ameliorating ptosis and/or improving facial appearance. Embodiments of the present invention include ointments and solutions that include one or more of the compounds travoprost, dorzolamide hydrochloride, and timolol maleate, alone or in combination, and/or one or more chemically-related compounds. These ointments and solutions are topically applied to eyelids in order to treat ptosis and/or to improve facial appearance. The ointments and solution may include, in addition to the above-mentioned active ingredients, stabilizers, buffering compounds, salts, carriers, viscosity enhancers, colorants, emollients, emulsifiers, bactericidal and germicidal agents, preservatives, and other ingredients that improve shelf life, application, consistency, and compatibility with topical application, among other things.

1. An ointment or solution used for treating ptosis and for cosmetic applications, the ointment or solution comprising:
a carrier; and
one or more of
travoprost,
dorzolamide hydrochloride,
timolol maleate,
unoprostone,
latanoprost, and
bimatoprost.
